(19) United States
(12) Patent Application Publication
(10) Pub. No.: US 2006/0094022 A1
(43) Pub. Date: May 4, 2006

(54) Array hybridization method including determination of completeness of restriction digest

(76) Inventors: Nicholas M. Sampas, Loveland, CO (US); Michael Thomas Barrett, Loveland, CO (US)

Correspondence Address: AGILENT TECHNOLOGIES, INC.;Intellectual Property Administration, Legal Department, DL429, P. O. Box 7599, Loveland, CO, 80537-0599, US

(21) Appl. No.: 10/978,273

(22) Filed: Oct. 30, 2004

(51) Int. Cl. C12Q 1/68; G06F 19/00

(52) U.S. Cl. 435/6; 702/20

(57) ABSTRACT

The present invention provides a method of performing an array hybridization analysis of a sample, including performing a restriction digest reaction on the sample, hybridizing the digested sample to the array, and interrogating the array. The array includes probe sets that provide for a determination of the extent of the restriction digest performed on the sample. Arrays including the probe sets are also described.

### FIELD OF INVENTION

The invention relates generally to bioarrays having polynucleotides bound to substrate and methods of using the bioarrays. More specifically, the invention relates to designing probe sets for use on bioarrays and methods of using bioarrays having such probe sets.

### BACKGROUND OF THE INVENTION

Polynucleotide arrays (such as DNA or RNA arrays) are known and are used, for example, as diagnostic or screening tools. Such arrays include regions of usually different sequence polynucleotides arranged in a predetermined configuration on a substrate. The arrays are “addressable” in that these regions (sometimes referenced as “array features”) have different predetermined locations (“addresses”) on a substrate of array. The polynucleotide arrays typically are fabricated on planar substrates either by depositing previously obtained biomolecules onto the substrate in a site specific fashion or by site specific in situ synthesis of the biomolecules upon the substrate.

The arrays, when exposed to a sample, will undergo a binding reaction with the sample and exhibit an observed binding pattern. This binding pattern can be detected upon interrogating the array. For example all target polynucleotides (for example, DNA) in the sample can be labeled with a suitable label (such as a fluorescent compound), and the label then can be accurately observed (such as by observing the fluorescence pattern) on the array after exposure of the array to the sample. Assuming that the different sequence polynucleotides were correctly deposited in accordance with the predetermined configuration, then the observed binding pattern will be indicative of the presence and/or concentration of one or more components of the sample. Techniques for scanning arrays are described, for example, in U.S. Pat. No. 5,763,870 and U.S. Pat. No. 5,945,679. Still other techniques useful for observing an array are described in U.S. Pat. No. 5,721,435.

The mapping of common genomic aberrations has been a useful approach to discovering cancer-related genes. Alterations in DNA copy number are characteristic of many cancer types and are thought to drive some cancer pathogenesis process. These alterations include large chromosomal gains and/or losses, as well as smaller scale amplifications and/or deletions. Genomic instability may trigger the over-expression or activation of oncogenes and the silencing of tumor suppressors and DNA repair genes. Local fluorescence in-situ hybridization-based techniques were used early on for measurement of alterations in DNA copy number.

A genome-wide measurement technique referred to as Comparative Genomic Hybridization (CGH) is currently used for identification of chromosomal alterations in cancer, e.g., see Balsara, et al., “Chromosomal Imbalances in Human Lung Cancer”, Oncogene, 21(45):6877-83, 2002: and Mertens, et al., “Chromosomal imbalance maps of malignant solid tumors: a cytogenetic survey of 3185 neoplasm”, Cancer Research, 57(13):2765-80,1997. Using CGH, differentially labeled tumor and normal DNA are co-hybridized to normal metaphases. Ratios between tumor and normal labels enable the detection of chromosomal amplifications and deletions of regions that may include oncogenes and tumor suppressive genes. This method has a limited resolution however, of only about 10-20 Mbp (mega base pairs). This amount of resolution provided is insufficient to enable a determination of the borders of the chromosomal changes or to identify changes in copy number of single genes and small genomic regions.

A refinement of CGH referred to as array CGH (aCGH) enables the determination of changes in DNA copy number of relatively small chromosomal regions. In the aCGH measurement technique, tumor and normal DNA are co-hybridized to a microarray of thousands of genomic clones of BAC, cDNA or oligonucleotide probes, e.g., see Pollack, et al., “Genome-wide analysis of DNA copy number changes using cDNA microarrays”, Nature Genetics, 23(1): 41-46, 1999; Pinkel, et al., High resolution analysis of DNA copy number variation using comparative genomic hybridization to microarrays”, Nature Genetics, 20(2): 207-211, 1998; and Hedenfalk, et al., “Molecular classification of familial non-brca1/brca2 breast cancer”, PNAS 100:2532, 2003. By using oligonucleotide arrays, the resolution provided can, in theory, be finer than the necessary to identify single genes.

aCGH is now widely used to measure copy number variations in cancer genomes and to detect chromosome abnormalities in clinical genetics. aCGH experiments typically involve interrogating an array with equal amounts of labeled target DNA (e.g., tumor and normal) in each channel. This allows relative measurements of signal intensities corresponding to binding of target DNA to the probes on the arrays. However, the quality of data obtained in an aCGH experiment can vary extensively even within the same platform.

The preparation of samples for aCGH, regardless of platform (cDNA, or BAC) or genome of interest (e.g., human or mouse), typically involves enzymatic preparation of sample of interest (Pollack, et al., PNAS 99:12963-68, 2002). For example, a widely used protocol involves the use of one or two frequent cutting restriction enzymes to prepare size-restricted template for subsequent labeling reactions prior to hybridization to the array. The advantage of these protocols is that optimal sizes of templates can be reproducibly generated based on the distribution of restriction site within a genome of interest.

During sample preparation it is desirable that templates be restriction digested to completion in each experiment. Restriction digestion enzymes can be inhibited by the presence of various contaminants, such as salts and organic reagents, present in a sample. These contaminants are often present in biological material (e.g., biopsies) as a result of common fixatives used in acquiring and archiving samples (e.g., formalin fixation and paraffin embedding), or as a result of extraction protocols that require organic reagents. Alternatively, samples can be over digested resulting in degraded templates that do not hybridize efficiently to the appropriate probe sequences on an array.

Therefore, there is a need to objectively determine the extent of template digestion in a quantitative manner.

### SUMMARY

The invention addresses the aforementioned deficiencies in the art, and provides novel methods for performing an array hybridization analysis of a sample. In various embodiments, the method includes performing a restriction digest reaction on the sample to yield a digested sample, then hybridizing the digested sample to an array. A probe series is included on the array; the probe series has at least one probe set, and each of the probe sets includes a junction probe and a cognate flanking probe. The array is interrogated to obtain a junction hybridization signal and a cognate flanking hybridization signal, and the junction hybridization signal and cognate flanking hybridization signal are compared to determine the extent of the restriction digest reaction.

In further embodiments, an array includes a first probe series, the first probe series comprises a plurality of probe sets. Each of the plurality of probe sets includes a junction probe and at least one flanking probe, and each of the plurality of probe sets is directed to a different restriction site.

Additional objects, advantages, and novel features of this invention shall be set forth in part in the descriptions and examples that follow and in part will become apparent to those skilled in the art upon examination of the following specifications or may be learned by the practice of the invention. The objects and advantages of the invention may be realized and attained by means of the instruments, combinations, compositions and methods particularly pointed out in the appended claims.

### BRIEF DESCRIPTION OF THE DRAWINGS

These and other features of the invention will be understood from the description of representative embodiments of the method herein and the disclosure of illustrative apparatus for carrying out the method, taken together with the Figures, wherein

FIG. 1A schematically illustrates a portion of a template, and illustrates the relationships of the flanking probes and junction probes to the template in particular embodiments.

FIG. 1B also illustrates a portion of a template, and illustrates the relationships of the flanking probes and junction probes to the template in particular embodiments.

FIG. 2 depicts a probe series for which at least 80% of the probes have a calculated melting temperature that fall within a 6 degrees Celsius range.

To facilitate understanding, identical reference numerals have been used, where practical, to designate corresponding elements that are common to the Figures. Figure components are not drawn to scale.

### DETAILED DESCRIPTION

Before the invention is described in detail, it is to be understood that unless otherwise indicated this invention is not limited to particular materials, reagents, reaction materials, manufacturing processes, or the like, as such may vary. It is also to be understood that the terminology used herein is for purposes of describing particular embodiments only, and is not intended to be limiting. It is also possible that methods recited herein may be carried out in any order of the recited events which is logically possible, as well as the recited order of events.

It must be noted that, as used in the specification and the appended claims, the singular forms “a,” “an” and “the” include plural referents unless the context clearly dictates otherwise. Thus, for example, reference to “a solid support” includes a plurality of solid supports. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

In this specification and in the claims that follow, reference will be made to a number of terms that shall be defined to have the following meanings unless a contrary intention is apparent.

The term “oligomer” is used herein to indicate a chemical entity that contains a plurality of monomers. As used herein, the terms “oligomer” and “polymer” are used interchangeably, as it is generally, although not necessarily, smaller “polymers” that are prepared using the functionalized substrates of the invention, particularly in conjunction with combinatorial chemistry techniques. Examples of oligomers and polymers include polydeoxyribonucleotides (DNA), polyribonucleotides (RNA), other nucleic acids that are C-glycosides of a purine or pyrimidine base, polypeptides (proteins), polysaccharides (starches, or polysugars), and other chemical entities that contain repeating units of like chemical structure.

The term “nucleic acid” and “polynucleotide” are used interchangeably herein to describe a polymer of any length composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length. Oligonucleotides are usually synthetic and, in many embodiments, are under 50 nucleotides in length.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “surface-bound polynucleotide” refers to a polynucleotide that is immobilized on a surface of a solid substrate, where the substrate can have a variety of configurations, e.g., a sheet, bead, or other structure. In certain embodiments, the collections of oligonucleotide target elements employed herein are present on a surface of the same planar support, e.g., in the form of an array.

The phrase “labeled population of nucleic acids”, or “labeled polynucleotides”, or other such language refers to mixture of nucleic acids that are detectably labeled, e.g., fluorescently labeled, such that the presence of the nucleic acids can be detected by assessing the presence of the label. The labeled population of nucleic acids is “made from” a chromosome source, the chromosome source is usually employed as template for making the population of nucleic acids. In particular embodiments, the sample that is hybridized on an array includes reference target and analyte target, wherein the reference target and the analyte target are differentially labeled.

The term “array” encompasses the term “microarray” and refers to an ordered array presented for binding to nucleic acids and the like.

An “array,” includes any one-dimensional, two-dimensional, substantially two-dimensional or three-dimensional arrangement of spatially addressable regions bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a front surface of the substrate. Depending upon the use, any or all of the arrays may be the same or different from one another and each may contain multiple spots or features. A typical array may contain one or more, including more than two, more than ten, more than one hundred, more than one thousand, more ten thousand features, or even more than one hundred thousand features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 and less than 1.5 mm, such as more than about 0.8 mm and less than about 1.2 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 mn.

Arrays can be fabricated using drop deposition from pulse-jets of either nucleic acid precursor units (such as monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

An array is “addressable” when it has multiple regions of different moieties (e.g., different oligonucleotide sequences) such that a region (i.e., a “feature” or “spot” of the array) at a particular predetermined location (i.e., an “address”) on the array will detect a particular sequence. Array features are typically, but need not be, separated by intervening spaces. In the case of an array in the context of the present application, the “population of labeled nucleic acids” will be referenced as a moiety in a mobile phase (typically fluid), to be detected by “surface-bound polynucleotides” which are bound to the substrate at the various regions. These phrases are synonymous with the terms “target” and “probe”, or “probe” and “target”, respectively, as they are used in other publications.

A “scan region” refers to a contiguous (preferably, rectangular) area in which the array spots or features of interest, as defined above, are found or detected. Where fluorescent labels are employed, the scan region is that portion of the total area illuminated from which the resulting fluorescence is detected and recorded. Where other detection protocols are employed, the scan region is that portion of the total area queried from which resulting signal is detected and recorded. For the purposes of this invention and with respect to fluorescent detection embodiments, the scan region includes the entire area of the slide scanned in each pass of the lens, between the first feature of interest, and the last feature of interest, even if there exist intervening areas that lack features of interest.

An “array layout” refers to one or more characteristics of the features, such as feature positioning on the substrate, one or more feature dimensions, and an indication of a moiety at a given location. “Hybridizing” and “binding”, with respect to nucleic acids, are used interchangeably.

The term “stringent assay conditions” as used herein refers to conditions that are compatible to produce binding pairs of nucleic acids, e.g., probes and targets, of sufficient complementarity to provide for the desired level of specificity in the assay while being incompatible to the formation of binding pairs between binding members of insufficient complementarity to provide for the desired specificity. Stringent assay conditions are the summation or combination (totality) of both hybridization and wash conditions.

A “stringent hybridization” and “stringent hybridization wash conditions” in the context of nucleic acid hybridization (e.g., as in array, Southern or Northern hybridizations) are sequence dependent, and are different under different experimental conditions. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions may affect the degree to which nucleic acids are specifically hybridized to complementary probes. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 7220 C. for about 15 minutes; or, a salt concentration of about 0.2×SSC at a temperature of at least about 50° C. or about 55° C. to about 60° C. for about 1 to about 20 minutes; or, multiple washes with a solution with a salt concentration of about 0.1×SSC containing 0.1% SDS at 20 to 50° C. for 1 to 15 minutes; or, equivalent conditions. Stringent conditions for washing can also be, e.g., 0.2×SSC/0.1% SDS at 42° C. In instances wherein the nucleic acid molecules are deoxyoligonucleotides (i.e., oligonucleotides), stringent conditions can include washing in 6×SSC/0.05% sodium pyrophosphate at 37° C. (for 14-base oligos), 48° C. (for 17-base oligos), 55° C. (for 20-base oligos), and 60° C. (for 23-base oligos). See Sambrook, Ausubel, or Tijssen (cited below) for detailed descriptions of equivalent hybridization and wash conditions and for reagents and buffers, e.g., SSC buffers and equivalent reagents and conditions.

A specific example of stringent assay conditions is rotating hybridization at 65° C. in a salt based hybridization buffer with a total monovalent cation concentration of 1.5M (e.g., as described in U.S. patent application Ser. No. 09/655,482 filed on Sep. 5, 2000, the disclosure of which is herein incorporated by reference) followed by washes of 0.5×SSC and 0.1×SSC at room temperature and 37° C.

Stringent hybridization conditions may also include a “prehybridization” of aqueous phase nucleic acids with complexity-reducing nucleic acids to suppress repetitive sequences. For example, certain stringent hybridization conditions include, prior to any hybridization to surface-bound polynucleotides, hybridization with Cot-1 DNA, or the like.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

The term “mixture”, as used herein, refers to a combination of elements, that are interspersed and not in any particular order. A mixture is heterogeneous and not spatially separable into its different constituents. Examples of mixtures of elements include a number of different elements that are dissolved in the same aqueous solution, or a number of different elements attached to a solid support at random or in no particular order in which the different elements are not especially distinct. In other words, a mixture is not addressable. To be specific, an array of surface bound polynucleotides, as is commonly known in the art and described below, is not a mixture of capture agents because the species of surface bound polynucleotides are spatially distinct and the array is addressable.

“Isolated” or “purified” generally refers to isolation of a substance (compound, polynucleotide, protein, polypeptide, polypeptide, chromosome, etc.) such that the substance comprises the majority percent of the sample in which it resides. Typically in a sample a substantially purified component comprises 50%, preferably 80%-85%, more preferably 90-95% of the sample. Techniques for purifying polynucleotides and polypeptides of interest are well known in the art and include, for example, ion-exchange chromatography, affinity chromatography, flow sorting, and sedimentation according to density.

The term “assessing” and “evaluating” are used interchangeably to refer to any form of measurement, and includes determining if an element is present or not. The terms “determining,” “measuring,” and “assessing,” and “assaying” are used interchangeably and include either or both of quantitative and qualitative determinations. Assessing may be relative or absolute. “Assessing the presence of” includes determining the amount of something present, as well as determining whether it is present or absent.

The acronym “CGH” refers to Comparative Genomic Hybridization.

The acronym “aCGH” refers to microarray-based CGH.

The term “aCGH array” refers to a microarray used to perform an aCGH experiment. Typically, an aCGH array or aCGH microarray is designed specifically for CGH measurements, in which case probes are designed to hybridize with genomic DNA. However, in some cases, a standard expression array can be used, since the DNA probes designed to measure RNA will also be complementary to the genomic DNA coding for those transcripts.

The term “sample” as used herein relates to a material or mixture of materials, typically, although not necessarily, in fluid form, containing one or more components of interest.

“Template” references a polynucleotide, typically from a genome of an organism. The sequence of the template is typically known, and probes may be designed using the known sequence. Template includes samples of polynucleotides isolated from the organism for analysis, e.g., analysis by array hybridization using an array having probes designed to specifically bind to the template.

“Known template sequence”, or “known sequence of genomic template”, references a polynucleotide for which sequence information is available, the sequence information typically being used to design probes for use on an array.

“Restriction site” references a site on a polynucleotide which a given restriction endonuclease will recognize and at which the restriction endonuclease may cut the polynucleotide (or has cut the polynucleotide). The “recognition sequence” is the polynucleotide sequence that a restriction endonuclease specifically recognizes before cutting the polynucleotide at the restriction site.

“Probe” references a polynucleotide immobilized to a substrate. The probe need not be limited to any particular length, but in particular embodiments the probe is at least about 10 nucleotides long, or at least about 15 nucleotides long, or at least about 20 nucleotide long, and the probe may be up to about 80 nucleotides long, or up to about 120 nucleotides long, or up to about 200 nucleotides long, or even longer. The probe is typically designed to be complementary to a template (or a portion of a template, i.e., a subsequence of the template). The phrase “directed to” describes a relationship between a particular polynucleotide sequence (e.g., a polynucleotide having the particular sequence of bases) and something that specifically recognizes or is recognized by that particular polynucleotide sequence (such as a complementary polynucleotide, a probe or target of an array, a restriction endonuclease). For example, a probe is directed to a target, a first polynucleotide is directed to a second polynucleotide that is complementary to the first polynucleotide, a restriction endonuclease is directed to the recognition sequence of the restriction endonuclease.

“Junction probe” references a probe having a sequence which bridges a restriction site (e.g., of a template) and which is complementary to the sequences immediately adjacent the restriction site. “Bridge site” references a site on a junction probe that directly corresponds to the restriction site of a complementary template, i.e. the bridge site is at the same position on the junction probe as the restriction site is at on the complementary template, wherein the complementary template is the template that the junction probe is directed to. In typical embodiments of the present invention, the bridge site of a junction probe typically is at or near the center of the junction probe, e.g. at a site between about 40% and about 60% of the distance along the junction probe (wherein the two ends of the junction probe are indicated as 0% and 100% of the distance along the junction probe, respectively). In certain embodiments of the present invention, the bridge site of the junction probe typically is at a site between about 30% and about 70%, or more typically is at a site between about 20% and about 80%, of the distance along the junction probe.

“Flanking probe” references a probe having a sequence complementary to a site that is proximal to a restriction site, provided that the flanking probe does not bridge the restriction site. The site that is complementary to the flanking probe (and is proximal to the restriction site) typically lies which a few hundred bases from the restriction site, e.g. typically within about 1000 bases, e.g. typically within about 100 bases, more typically within about 200 bases, still more typically within 300 bases, even more typically within about 400 bases, yet more typically within about 500 bases, yet still more within about 600 bases from the restriction site. In certain embodiments, the flanking probe is complementary to a site that lies within about 20 bases of the restriction site, or within about five bases of the restriction site. In particular embodiments, the flanking probe is complementary to a site that terminates at the restriction site, i.e. there are no intervening bases between the complementary sequence and the restriction site (in other words, the flanking probe is directed to a sequence directly adjacent the restriction site). In certain embodiments the flanking probe overlaps the junction probe, but in other embodiments the flanking probe does not overlap the junction probe. By “overlap” it is meant that the cognate flanking probe is directed to a template sequence which is partly overlapped by the sequence that the respective junction probe is directed to, i.e. the junction probe and the cognate flanking probe have sequence identity over a portion of their sequence.

“Primary probes” are probes directed to known sequences of genomic template, wherein the array does not include junction probes directed to sequences within about 1000 bases (e.g. within about 600, 500, 400, 300 bases) from the known sequences of genomic template that the primary probes are directed to.

“Probe set” references a group of at least two probes corresponding to a given restriction site of a template, i.e. the probes of the probe set are designed to bind to a given polynucleotide (e.g. a known template sequence) having a sequence that includes the restriction site, in which case, for convenience herein, the probe set is said to be “directed to” the restriction site. The probe set includes a junction probe and at least one flanking probe. In some embodiments the probe set includes the junction probe and two or more flanking probes, e.g. three, four, five, or more flanking probes. In some embodiments, the probe set may include two, three, four, five or more junction probes; in typical embodiments, a probe set will have one junction probe.

“Probe series” references a plurality of probe sets; wherein each of the plurality of probe sets corresponds to a different restriction site of the template, wherein each of the restriction sites may be cleaved by the same restriction endonuclease. A probe series thus includes plurality of junction probes, plus one or more flanking probes corresponding to each junction probe (i.e. from the same probe set as each junction probe). In particular embodiments of the present invention a probe series will have at least 5 different probe sets, e.g. at least 5 different junction probes and at least one flanking probe for each of the different junction probes. In certain embodiments of the present invention a probe series will have at least 10 different probe sets, e.g. at least 10 different junction probes and at least one flanking probe for each of the different junction probes. In some embodiments of the present invention a probe series will have at least 20 different probe sets, e.g. at least 20 different junction probes and at least one flanking probe for each of the different junction probes. In some embodiments of the present invention a probe series will have at least 40 different probe sets, e.g. at least 40 different junction probes and at least one flanking probe for each of the different junction probes. In certain embodiments of the present invention a probe series may have up to about 200 different probe sets or more, e.g. up to about 150 probe sets, up to about 120 probe sets, up to about 100 probe sets, up to about 80 probe sets, or more.

“Junction hybridization signal” references information obtained by interrogating (reading) an array at an array feature which has a junction probe. The junction hybridization signal is typically a quantitative measure of hybridization of target from the sample to the junction probe on the array, e.g. fluorescence intensity. In some embodiments, the junction hybridization signal may be a qualitative measure of hybridization of target from sample to the junction probe on the array. In certain embodiments, the junction hybridization signal is an absolute measure of hybridization of target from the sample to the junction probe on the array, e.g. an absolute measure of fluorescence intensity. In particular embodiments, the junction hybridization signal is a relative measure of hybridization of target from the sample to the junction probe on the array, e.g. a measure of fluorescence intensity relative to a control or other signal, e.g. a fluorescence measurement from a second channel in a “two-color” measurement of binding to a junction probe. Typical two-color measurements are known in the art especially relating to aCGH.

“Flanking hybridization signal” references information obtained by interrogating (reading) an array at an array feature which has a flanking probe. The flanking hybridization signal is typically a quantitative measure of hybridization of target from the sample to the flanking probe on the array, e.g. fluorescence intensity. In some embodiments, the flanking hybridization signal may be a qualitative measure of hybridization of target from the sample to the flanking probe on the array. In certain embodiments, the flanking hybridization signal is an absolute measure of hybridization of target from the sample to the flanking probe on the array, e.g. an absolute measure of fluorescence intensity. In particular embodiments, the flanking hybridization signal is a relative measure of hybridization of target from the sample to the flanking probe on the array, e.g. a measure of fluorescence intensity relative to a control or other signal, e.g. a fluorescence measurement from a second channel in a “two-color” measurement of binding to a flanking probe. A “cognate flanking hybridization signal” is a flanking hybridization signal obtain by interrogating the array at an array feature which has a cognate flanking probe; furthermore, interrogating an array at an array feature which has a junction probe in the same probe set as the cognate flanking probe provides the respective junction hybridization signal.

The term “cognate” is used to refer to members of the same probe set, e.g. a junction probe and a cognate flanking probe. For example, for a junction probe having a sequence which bridges a restriction site (e.g. of a template) and which is complementary to the sequences immediately adjacent the restriction site, a cognate flanking probe is a flanking probe having a sequence complementary to a site that is proximal to that restriction site. A junction probe that is a member of the same probe set as a flanking probe may be referred to herein as a “respective” junction probe of the flanking probe, or the flanking probe's respective junction probe.

As used herein “site” may reference a relatively short portion of a polynucleotide, e.g. a portion of the polynucleotide that is less than about 200 bases long. For example, “site” may reference the portion of a polynucleotide along which a complementary probe will bind. As used herein “site” may reference a discrete location between two adjacent bases in a sequence, for example, a bridge site or a restriction site. As used herein “site” may reference a location or a position, such as a position on a substrate. Context will determine the intended definition.

“Complementary” references a property of specific binding between polynucleotides based on the sequences of the polynucleotides. As used herein, polynucleotides are complementary if they bind to each other in a hybridization assay under stringent conditions, e.g. if they produce a given or detectable level of signal in a hybridization assay. Portions of polynucleotides are complementary to each other if they follow conventional base-pairing rules, e.g. A pairs with T (or U) and G pairs with C. “Complementary” includes embodiments in which there is an absolute sequence complementarity, and also embodiments in which there is a substantial sequence complementarity. “Absolute sequence complementarity” means that there is 100% sequence complementarity between a first polynucleotide and a second polynucleotide, i.e. there are no insertions, deletions, or substitutions in either of the first and second polynucleotides with respect to the other polynucleotide (over the complementary region). Put another way, every base of the complementary region may be paired with its complementary base, i.e. flowing normal base-pairing rules. “Substantial sequence complementarity” permits one or more relatively small (less than 10 bases, e.g. less than 5 bases, typically less than 3 bases, more typically a single base) insertions, deletions, or substitutions in the first and/or second polynucleotide (over the complementary region) relative to the other polynucleotide. The region that is complementary between a first polynucleotide and a second polynucleotide (e.g. a target and a probe) is typically at least about 10 bases long, more typically at least about 15 bases long, still more typically at least about 20 bases long, or at least about 25 bases long. The region that is complementary between a first polynucleotide and a second polynucleotide (e.g. target and a probe) may be up to about 200 bases long, or more typically up to about 120 bases long, more typically up to about 100 bases long, still more typically up to about 80 bases long, yet more typically up to about 60 bases long, more typically up to about 45 bases long.

“Upstream” as used herein refers to the 5′ direction along the template. “Downstream” refers to the 3′ direction along the template. Hence, a probe downstream of a restriction site of the template is located at (or is complementary to) a sequence of the template that is in the 3′ direction from the restriction site along the template. A “downstream flanking probe” is directed to a sequence in the 3′ direction along the template from its respective restriction site. Similarly, an “upstream flanking probe” is directed to a sequence in the 5′ direction along the template from its respective restriction site.

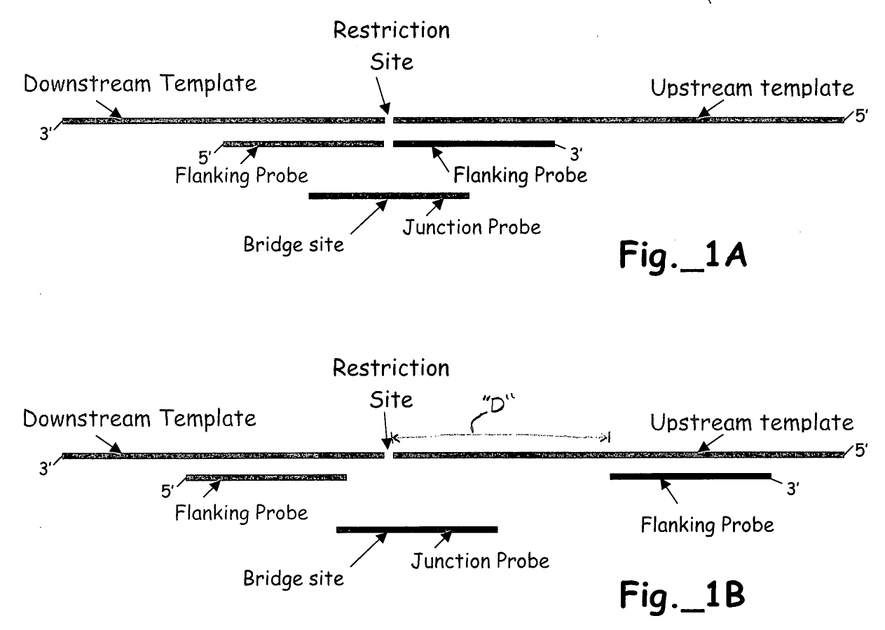

Terms used in describing the invention are illustrated in FIG. 1A and FIG. 1B. In FIG. 1A, the template is cut at the restriction site, and the upstream (in the 5′ direction) and downstream (in the 3′ direction) portions of the template are shown. In FIG. 1A, the junction probe and the flanking probes (including an upstream flanking probe and a downstream flanking probe) are aligned along the template to illustrate the sites of the template that they are directed to (are complementary to). Both flanking probes are adjacent the restriction site and are overlapping the junction probe. The junction probe and the two flanking probes together make up a probe set that is directed to the restriction site shown in the Figure.

In comparison, in FIG. 1B, the flanking probes are complementary to different sites of the template, which are no longer adjacent the restriction site. The junction probe in FIG. 1B also is positioned such that the bridge site is shifted in the junction probe, and there is only a small overlap with one of the flanking probes.

Although much of the description herein is directed at aCGH applications, the invention is not limited to methods of array hybridization for aCGH applications. Rather, the methods (and compositions) provided in accordance with the present invention relate more generally to array hybridization methods having a sample preparation step that includes a restriction digest reaction. In typical embodiments, the invention provides a method of performing an array hybridization analysis of a sample, wherein, in particular embodiments, the method provides for determining the extent of a restriction digest reaction performed as a sample preparation step.

The method includes performing a restriction digest reaction on a sample to yield a digested sample, and hybridizing the digested sample to an array. The array used in the method includes a probe series, the probe series comprising at least one probe set, each of the least one probe sets comprising a junction probe and a cognate flanking probe. After hybridizing the digested sample to an array, the array is interrogated to obtain a junction hybridization signal and a cognate flanking hybridization signal. The junction hybridization signal and cognate flanking hybridization signal are compared to determine the extent of the restriction digest reaction.

The restriction digest reaction is conducted according to well known methods. The reaction typically is performed by contacting a sample with one or more restriction endonucleases in solution under conditions sufficient to result in cleavage of DNA having a sequence which includes an appropriate recognition sequence for the restriction endonuclease. Relevant methods are described in Ausubel et al, Short Protocols in Molecular Biology, 3rd ed., Wiley & Sons, 1995 and Sambrook et al, Molecular Cloning: A Laboratory Manual, Third Edition, 2001 Cold Spring Harbor, N.Y. The selection of the endonuclease typically will depend on the design of the array, i.e. the design of the probe sets on the array (or the probe series). It should be apparent that the design and selection of the probe sets on the arrays used in the array hybridization methods of the invention is tied to the particular restriction endonuclease(s) used (or intended to be used) in the restriction digest reaction.

Standard hybridization techniques (using stringent hybridization conditions) are used to hybridize a labeled sample to a nucleic acid array. Suitable methods are described in references describing CGH techniques (Kallioniemi et al., Science 258:818-821 (1992) and WO 93/18186). Several guides to general techniques are available, e.g., Tijssen, Hybridization with Nucleic Acid Probes, Parts I and II (Elsevier, Amsterdam 1993). For a descriptions of techniques suitable for in situ hybridizations see, Gall et al. Meth. Enzymol., 21:470-480 (1981) and Angerer et al. in Genetic Engineering: Principles and Methods Setlow and Hollaender, Eds. Vol 7, pgs 43-65 (plenum Press, New York 1985). See also U.S. Pat. Nos.: 6,335,167; 6,197,501; 5,830,645; and 5,665,549; the disclosures of which are herein incorporate by reference. Hybridizing the sample to the array is typically performed under stringent hybridization conditions, as described herein and as known in the art. Selection of appropriate conditions, including temperature, salt concentration, polynucleotide concentration, time(duration) of hybridization, stringency of washing conditions, and the like will depend on experimental design, including source of sample, identity of probes, degree of complementarity expected, and are within routine experimentation for those of ordinary skill in the art to which the invention applies.

Following hybridization, the array-surface bound polynucleotides are typically washed to remove unbound and not tightly bound labeled nucleic acids. Washing may be performed using any convenient washing protocol, where the washing conditions are typically stringent, as described above.

Following hybridization and washing, as described above, the hybridization of the labeled nucleic acids to the targets is then detected using standard techniques so that the surface of immobilized targets, e.g., the array, is interrogated, or read. Reading the resultant hybridized array may be accomplished by illuminating the array and reading the location and intensity of resulting fluorescence at each feature of the array to detect any binding complexes on the surface of the array. For example, a scanner may be used for this purpose, which is similar to the AGILENT MICROARRAY SCANNER available from Agilent Technologies, Palo Alto, Calif. Other suitable devices and methods are described in U.S. patent applications: Ser. No. 09/846125 “Reading Multi-Featured Arrays” by Dorsel et al.; and U.S. Pat. No. 6,406,849. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). In the case of indirect labeling, subsequent treatment of the array with the appropriate reagents may be employed to enable reading of the array. Some methods of detection, such as surface plasmon resonance, do not require any labeling of nucleic acids, and are suitable for some embodiments.

Results from the reading or evaluating may be raw results (such as fluorescence intensity readings for each feature in one or more color channels) or may be processed results (such as those obtained by subtracting a background measurement, or by rejecting a reading for a feature which is below a predetermined threshold, normalizing the results, and/or forming conclusions based on the pattern read from the array (such as whether or not a particular target sequence may have been present in the sample, or whether or not a pattern indicates a particular condition of an organism from which the sample came).

In certain embodiments, results from interrogating the array are used to assess the level of binding of the population of labeled nucleic acids to probes on the array. The term “level of binding” means any assessment of binding (e.g. a quantitative or qualitative, relative or absolute assessment) usually done, as is known in the art, by detecting signal (i.e., pixel brightness) from a label associated with the sample nucleic acids, e.g. the digested sample is labeled. The level of binding of labeled nucleic acid to probe is typically obtained by measuring the surface density of the bound label (or of a signal resulting from the label).

In certain embodiments, a surface-bound polynucleotide may be assessed by evaluating its binding to two populations of nucleic acids that are distinguishably labeled. In these embodiments, for a single surface-bound polynucleotide of interest, the results obtained from hybridization with a first population of labeled nucleic acids may be compared to results obtained from hybridization with the second population of nucleic acids, usually after normalization of the data. The results may be expressed using any convenient means, e.g., as a number or numerical ratio, etc.

Results from reading the array include a junction hybridization signal and a cognate flanking hybridization signal, typically for each probe set on the array. In particular embodiments, a probe series includes at least 5 probe sets, e.g. at least 10, at least 20, or at least 40 or more probes sets. Interrogating the array provides a junction hybridization signal and a cognate flanking hybridization signal for each of the at least 5 probe sets. In particular embodiments, the probes within each set are compared, and results for all such comparisons are evaluated to determine the extent of the restriction digest reaction. In certain embodiments, the process of comparing may include discarding one or more probe sets (e.g. discarding or ignoring the signals from fewer than 20% of probe sets) which are determined to present anomalous data, relative to the remaining data. In certain embodiments, rather than discarding data, the anomalous data sets are weighted less than the remaining data in determining the extent of the restriction digest reaction.

In an exemplary embodiment, comparing a junction hybridization signal and a cognate flanking hybridization signal to determine the extent of restriction digent reaction may include making a “call” based on each junction hybridization signal and a cognate flanking hybridization signal. For each flanking probe a call can be made by comparing the flanking hybridization signal to the junction hybridization signal and determining whether the probe set confirms an effective degree of hybridization. In its simplest form one can simply compare the flanking hybridization signal (or an averaged signal) to the respective junction hybridization signal, and if the junction hybridization signal is significantly lower than the flanking hybridization signal then the “call” is that the site is effectively cut. If multiple features exist for either the flanking probe, or the junction probe or both then more sophisticated tests can be done to test whether the flanking probe features share the same distribution as the junction probe's features. If a given junction probe has multiple cognate flanking probes then a statistical test can be performed to determine whether these probes share the same distribution. One example of such a test is the t-test which gives the P-value for the significance of the separation of the distributions, where the P-value is the statistical probability that the data sets represent the same distribution. A specific example of the t-test is Student's 2-tailed t-test. In various embodiments a call can be made on a probe-by-probe basis or on a probe set by probe set basis.

In other embodiments a voting test is used: When a series of probe sets are utilized, then a statistical test can be performed where the ensemble of probe sets are considered. A simple test is simply a count of how many flanking probes have signals that are higher than their respective junction probe.

Because there may be signal biases associated with signals for different probes with different sequences, and because it is not possible to reliably predict these biases, typically an experimental set of measurements are done to determine how many votes are considered statistically significant enough to characterize the digest as being effectively completed, or to give a quantitative measure of the extent of completion of the digest. Thus a calibration series of experiments with different experimental conditions that limit the effectiveness of the restriction enzyme in cutting the template are carried out. An analysis of these experiments would yield a calibration curve or response curve that would provide the relationship between the voting score and the degree of digestion of the target sequences, and perhaps degrees of confidence of each point within the curve. With this standard curve information it is possible to characterize the degree of fragmentation of the sample. Such curves would be performed for each color (dye, or emission filter, for fluorescence detection) of the detection system, as well as for each enzyme that may be used in the digest. By having different probe sets for each enzyme where multiple enzymes are used, it is possible to determine the effectiveness of each enzyme in the restriction digest reaction.

In certain embodiments, arrays having probe sets as described here are provided. Such an array includes a first probe series present on a substrate, the first probe series comprising a plurality of probe sets, each of the plurality of probe sets comprising a junction probe and at least one flanking probe, each of the plurality of probe sets directed to a different restriction site.

In particular embodiments, all of the flanking probes are complementary to sites of the template that are within about 1000 bases, e.g. is less than 600 bases, less than 500 bases, less than 400 bases, less than 300 bases, less than 200 bases, or less than about 100 bases from the respective restriction site that the probe set is directed to. This is illustrated in FIG. 1B, showing the flanking probe at a distance “D” upstream of the junction probe. In particular embodiments, at least one flanking probe of each probe set is directed to a sequence that is within about 1000 bases, e.g. is less than 600 bases, less than 500 bases, less than 400 bases, less than 300 bases, less than 200 bases, or less than about 100 bases from the restriction site that the probe set is directed to.

As also illustrated in FIG. 1A and FIG. 1B, the probe set may include an upstream flanking probe and a downstream flanking probe. In particular embodiments, each probe set comprises at least one upstream flanking probe and at least one downstream flanking probe. In particular embodiments, at least one flanking probe of each probe set overlaps the junction probe from the same probe set.

FIG. 1A illustrates that the flanking probes of the probe set are directed to a sequence directly adjacent the restriction site that the probe set is directed to. Accordingly, in certain embodiments of an array provided by the present invention, at least one of the at least one flanking probes of each probe set is directed to a sequence directly adjacent the restriction site that the probe set is directed to.

In addition to one or more probe series (each including one or more probe sets) described above, an array in accordance with the present invention will typically have primary probes directed to directed to known sequences of genomic template, wherein the array does not include junction probes directed to sequences within about 1000 bases (e.g. within about 600, 500, 400, 300 bases) from the known sequences of genomic template that the primary probes are directed to. In other words, primary probes are probes that are not directed to any part of the genomic sequence that has a junction probe directed to a sequence within about 1000 bases. In certain embodiments, the number of primary probes on the array is at least about 5 times the total number of junction probes on the array and is less than about 5000 times the total number of junction probes on the array. In particular embodiments, the number of primary probes on the array is at least about 5 times the total number of junction probes on the array and is less than about 100 times the total number of junction probes on the array. And in other embodiments, the number of primary probes on the array is at least about 100 times the total number of junction probes on the array and is less than about 5000 times the total number of junction probes on the array.

In certain embodiments, the array includes at least two probe series, each probe series directed to a different restriction endonuclease. In some such embodiments, the array comprises a second probe series, the second probe series comprising a plurality of probe sets, each of the plurality of probe sets of the second probe series comprising a junction probe and at least one flanking probe, each of the plurality of probe sets of the second probe series directed to a different restriction site which may be cleaved by a second restriction endonuclease, and the probe sets of the first probe series are directed to restriction sites which may be cleaved by a first restriction endonuclease which is different from the second restriction endonuclease.

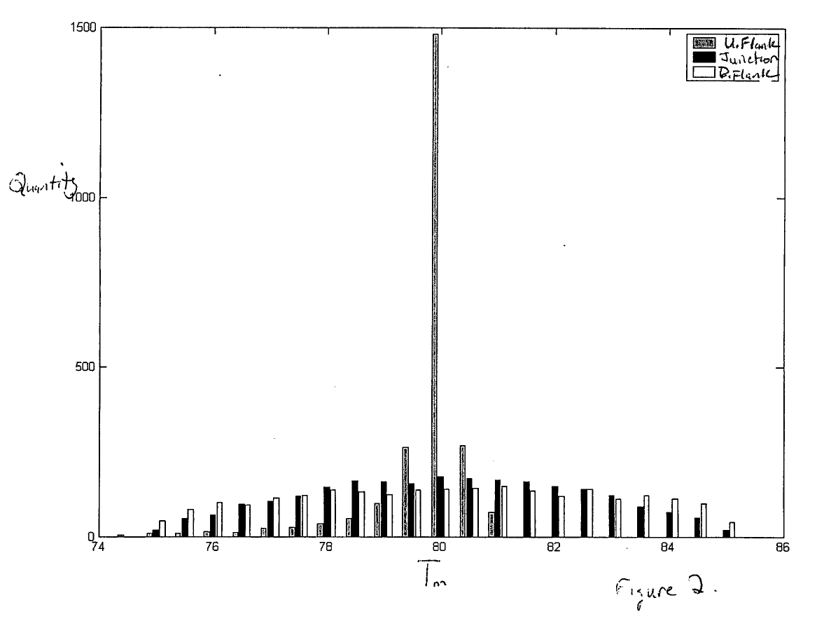

In selecting probes to make up to probe sets, it was useful to use a computational algorithm to produce a calculated melting temperature for each probe. Probe sets or even probe series that have a narrow melting temperature range may be particularly suited for some applications of array hybridization analysis. A nearest neighbor analysis that adjusted for mismatches in the probe sequences was used to generate the calculated melting temperatures. In an embodiment with no mismatches, a simpler nearest neighbor algorithm can be used. Software methods for calculating melting temperatures are well developed, and such may be obtained from various commercial or academic sources. Some commercial sources for software include Alkami Biosystems, Molecular Biology Insights, PREMIER Biosoft International, IntelliGenetics Inc., Hitachi Inc., DNA Star, Advanced American Biotechnology and Imaging. Various references have described melting temperature calculations, including Breslauer et al., “Predicting DNA duplex stability from the base sequence”, Proc Natl Acad Sci. 83(11): 3746-3750 (1986); Sugimoto et al., “Improved thermodynamic parameters and helix initiation factor to predict stability of DNA duplexes” Nucleic Acids Research 24: 4501 (1996); Xia et al., “Thermodynamic parameters for an expanded nearest-neighbor model for formation of RNA duplexes with Watson-Crick base pairs” Biochemistry. 1998 October 20;37(42): 14719-35; and references therein. A tentative probe series was identified for which at least 80% of the probes had a calculated melting temperature that fell within a 6 degrees Celsius range (see FIG. 2). This was accomplished by first identifying restriction sites in the genomic template of interest and selecting potential flanking probes adjacent to the restriction sites. These potential flanking probes were then screened to eliminate those which had a melting temperature outside the desired range and also to eliminate those with repetitive sequences. The remaining probes were then screened to find potential junction probes corresponding to the potential flanking probes which had melting temperatures in the desired range and did not have repetitive sequences. We also selected for potential junction probes with bridge sites very close to the middle of the potential junction probes. Similarly, we then searched for probe sets from those remaining where at least about 80% of the flanking probes and the junction probes fell in the range of 77-83 degrees. A histogram of the resulting probe series is shown in FIG. 2. A final probe series may be selected from the potential probe sets, e.g. based on sequence, GC content, AT content, location in the genome (e.g. near sites of interest), or based on empirical performance in use, or based on other appropriate factors. Therefore, in certain embodiments, the calculated melting temperatures of at least about 80% of the flanking probes and the junction probes on an array fall within a range of about 6 degrees Celsius. In certain such embodiments, the calculated melting temperature of each probe is obtained using a nearest neighbor analysis algorithm and the genomic template sequence that the probe is directed to, including any insertions, deletions, or substitutions. It is further noted that the particular methodology used to select probe sets is illustrative only, and should not be interpreted to limit the scope of the invention beyond the limitations set forth in the claims, below.

The arrays described above, in any of the various embodiments described, may be employed in the methods of performing an array hybridization described herein.

While the foregoing embodiments of the invention have been set forth in considerable detail for the purpose of making a complete disclosure of the invention, it will be apparent to those of skill in the art that numerous changes may be made in such details without departing from the spirit and the principles of the invention. Accordingly, the invention should be limited only by the following claims.

All patents, patent applications, and publications mentioned herein are hereby incorporated by reference in their entireties.

What is claimed is:

1. A method of performing an array hybridization analysis of a sample, the method comprising:
a) performing a restriction digest reaction on the sample to yield a digested sample,
b) hybridizing the digested sample to an array, wherein the array comprises a probe series, the probe series comprising at least one probe set, each of the at least one probe sets comprising a junction probe and a cognate flanking probe,
c) interrogating the array to obtain a junction hybridization signal and a cognate flanking hybridization signal, and
d) comparing the junction hybridization signal and cognate flanking hybridization signal to determine the extent of the restriction digest reaction.

2. The method of claim 1, wherein the probe series comprises at least 5 probe sets, wherein interrogating the array provides a junction hybridization signal and a cognate flanking hybridization signal for each of the at least 5 probe sets, and wherein comparing comprises comparing the junction hybridization signal and cognate flanking hybridization signal for each of the at least 5 probe sets to determine the extent of the restriction digest reaction.

3. The method of claim 2, wherein comparing includes discarding one or more outlying junction hybridization signals and the respective cognate flanking signals and determining the extent of the restriction digest reaction with the remaining junction hybridization signals and cognate flanking hybridization signals.

4. The method of claim 2, wherein comparing includes weighting one or more outlying junction hybridization signals and the respective cognate flanking signals less than the remaining junction hybridization signals and cognate flanking hybridization signals.

5. The method of claim 1, wherein interrogating comprises illuminating the array and detecting the location and intensity of resulting fluorescence at multiple features of the array.

6. The method of claim 1, wherein the restriction digest reaction comprises contacting the sample with at least two restriction endonucleases, and the array comprises a probe series for each of the at least two restriction endonucleases.

7. The method of claim 6, wherein each probe series comprises at least one probe set, each probe set comprising a junction probe and at least one flanking probe.

8. The method of claim 1, wherein the junction probe of each of the at least one probe sets has a bridge site at a position between about 30% and about 70% of the distance along the junction probe.

9. The method of claim 1, wherein the array further comprises primary probes directed to known sequences of genomic template, wherein the array does not include junction probes directed to sequences within about 1000 bases (600, 500, 400, 300) from the known sequences of genomic template that the primary probes are directed to.

10. The method of claim 9, wherein the number of primary probes on the array is at least about 5 times the total number of junction probes on the array and is less than about 5000 times the total number of junction probes on the array.

11. The method of claim 9, wherein the number of primary probes on the array is at least about 5 times the total number of junction probes on the array and is less than about 100 times the total number of junction probes on the array.

12. The method of claim 9, wherein the number of primary probes on the array is at least about 100 times the total number of junction probes on the array and is less than about 5000 times the total number of junction probes on the array.

13. The method of claim 1, wherein the array hybridization analysis is adapted to provide a measure of copy number variation in the sample.

14. The method of claim 1, wherein the sample includes reference target and analyte target, wherein the reference target and the analyte target are differentially labeled.

15. The method of claim 1, wherein hybridizing is performed under stringent conditions.

16. The method of claim 1, wherein the array comprises at least 5 probe sets.

17. The method of claim 16, wherein each probe set comprises at least two cognate flanking probes for each junction probe.

18. The method of claim 17, wherein the least two cognate flanking probes for each junction probe include an upstream flanking probe and a downstream flanking probe.

19. The method of claim 1, wherein the junction probe of each probe sets bridges a restriction site of a known template sequence and is complementary to the know template sequences immediately adjacent the restriction site, wherein the restriction digest reaction can cut at the restriction site, further wherein the cognate flanking probe of each probe set is directed to a portion of the known template sequence that is within about 1000 bases of the restriction site of the known template sequence.

20. The method of claim 19, wherein the cognate flanking probe of each probe set is directed to a portion of the known template sequence that is adjacent the restriction site of the known template sequence.

21. The method of claim 1, wherein the method is performed in conjunction with an array CGH assay.

22. An array comprising a first probe series, the first probe series comprising a plurality of probe sets, each of the plurality of probe sets comprising a junction probe and at least one flanking probe, each of the plurality of probe sets directed to a different restriction site.

23. The array of claim 22, wherein the at least one flanking probe of each probe set is directed to a sequence that is within about 1000 bases from the restriction site that the probe set is directed to.

24. The array of claim 22, wherein the at least one flanking probe of each probe set comprises at least one upstream flanking probe and at least one downstream flanking probe.

25. The array of claim 22, wherein at least one of the at least one flanking probes of each probe set is directed to a sequence directly adjacent the restriction site that the probe set is directed to.

26. The array of claim 22, wherein at least one of the at least one flanking probes of each probe set overlaps the at least one junction probe from the same probe set.

27. The array of claim 22, wherein in certain embodiments, the calculated melting temperatures of at least about 80% of the flanking probes and the junction probes on an array fall within a range of about 6 degrees Celsius.

28. The array of claim 22, wherein the array further comprises a second probe series, the second probe series comprising a plurality of probe sets, each of the plurality of probe sets of the second probe series comprising a junction probe and at least one flanking probe, each of the plurality of probe sets of the second probe series directed to a different restriction site which may be cleaved by a second restriction endonuclease, and the probe sets of the first probe series are directed to restriction sites which may be cleaved by a first restriction endonuclease which is different from the second restriction endonuclease.
